(19) United States
(12) Patent Application Publication
(10) Pub. No.: US 2023/0066947 A1
(43) Pub. Date: Mar. 2, 2023

(54) Method for the augmentation of substance abuse therapies using cannabinoid formulations

(71) Applicant: Max Hammond, Olive Hill, KY (US)

(72) Inventor: Max Hammond, Olive Hill, KY (US)

(21) Appl. No.: 17/394,398

(22) Filed: Aug. 4, 2021

(51) Int. Cl. A61K 31/352; A61K 31/192; A61K 31/05; A61K 31/137; A61K 31/485

(52) U.S. Cl. CPC A61K 31/352; A61K 31/192; A61K 31/05; A61K 31/137; A61K 31/485

(57) ABSTRACT

This disclosure relates to the use of preparations of CBDA to reduce the instances of relapse during addiction recovery and to increase its bioavailability using cyclodextrin. Other preparations are disclosed wherein decarboxylated cannabinoids and specific species of THC are also effective. The preparations may be used alone, to augment treatment following suboxone or methadone therapies, of utilized post-treatment.

### CROSS-REFERENCE TO RELATED APPLICATIONS

This application claims priority from U.S. Provisional Patent Application 63/061,176 filed on Aug. 4, 2020.

### TECHNICAL FIELD

The disclosures herein are associated with the treatment of substance abuse disorder and more specifically with using CBDA having enhanced bioavailability to reduce the severity of withdrawal symptoms in conjunction with addiction recovery therapies, including treatment with suboxone or methadone, to reduce instances of relapse.

### BACKGROUND

The formulations and methods of use of the present application relates generally the use of cannabinoids to improve outcomes in addiction recovery. More specifically, the application relates to formulations of cannabinoids coupled with an agent to increase bioavailability or the orally ingested product and their use in improving therapeutic outcomes in the treatment of substance abuse disorders.

Substance use disorder (SUD)—commonly referred to as addiction—is a medical illness with altered behavioral, cognitive, physical, neurobiological, and affective functions associated with compulsive and repeated use of addictive substance(s), whether legal or illegal. Regardless of the differences among the addictive substances, SUDs share common neurobehavioral characteristics, including the progression of the three addiction stages (intoxication→withdrawal→craving) and dysregulation of the neurobiological systems associated with reward, stress, emotion, and executive functions. Addiction causes millions of years of life lost because of premature death and is also among the leading causes of life with disability worldwide, including both developing and developed countries. Alcohol addiction, as an example, is a leading risk factor for deaths globally. In the United States, it is estimated that each year, over 88,000 people die from alcohol related causes. Other drug-overdose deaths have increased by more than threefold in the United States since 1999, resulting in more than 70,000 deaths in 2017. Based on the 2018 study by the Substance Abuse and Mental Health Services Administration (SAMHSA), in the United States alone, there are more than 16 million heavy alcohol drinkers, 27 million daily smokers, and more than 50 million illicit drug users, including more than 10 million people who misuse opioids. However, only about 10% of those who needed treatment for SUDs received treatments in 2018. Although there are effective medications—except for cocaine addiction—and other treatment options, the effectiveness of SUD treatment remains inadequate, as extensively reviewed by the leading experts. According to the 2016 United States Surgeon General's Report, more than 60% of those who received addiction treatments in the United States relapsed within a year, which highlights the challenges in sustaining recovery (i.e., maintaining long-term drug abstinence and well-being). Despite decades of scientific research and the high economic cost (estimated at $740 billion a year in the United States alone), treatment outcomes and recovery from SUDs continue to be very limited. Other studies have placed the relapse rate in excess of 90%.

Scientific studies on addiction have led to the development of a number of medications for pharmacological interventions, along with other non-pharmacotherapies including behavioral, cognitive, and social interventions (see the comprehensive reviews in this collection). These intervention methods have been applied in treating SUDs such as alcohol, nicotine, and opioid use disorders. Unfortunately, there are no targeted and effective medications for treating cocaine addiction at the present time, due to its complex effect on the central nervous system (CNS—the brain and spinal cord) and difficulty in identifying medication targets. Even for SUDs with validated treatments, their effectiveness is complicated by many factors related to the nature of the illness, particularly for people with severe SUDs. For instance, regardless of etiology, SUDs affect not only the brain but also other systems and vital organs including the liver, lungs, and the cardiovascular and digestive systems. Misused substances can induce epigenetic changes with widespread downstream biological consequences and alter the functioning of the immune and endocrine systems. Moreover, each substance may affect these systems differently and interactively in polysubstance use.

The endocannabinoid system (ECS) is the endogenous body system tasked with the maintenance and restoration of homeostasis utilizing cannabinoids and cannabinoid receptors. Homeostasis is the stability of the body's internal environment. When homeostasis is affected by injury or infection, the ECS attempts to return the body to a condition of homeostasis by regulating processes such as the inflammatory response.

Endocannabinoids are, fundamentally, cannabinoids produced by the body. The ECS has been called the master regulator in the human body in that it utilizes endocannabinoids to ensure homeostasis. Endocannabinoids are produced by the body as needed when properly nourished and maintained and are broken down by enzymes (e.g., fatty acid amid hydrolase and monoacylglycerol) after they have carried out their function. Obstacles to the production or breaking down of endocannabinoids can impair homeostasis. Endocannabinoid deficiency is a condition believed to result from low endocannabinoid levels or an ECS dysfunction. ECS dysfunction has been linked to several ailments which lack a definitive cause.

The ECS consists of cannabinoid receptors (e.g., CB1, CB2), the endogenous ligands that bind to these cannabinoid receptors [e.g., anandamide and 2-arachidonoylglycerol (2-AG)], and enzymes for their biosynthesis and degradation [e.g., fatty acid amide hydrolase (FAAH) and monoacylglyrecol lipase (MAGL)]. CB1 receptors (CB1R) are found throughout the peripheral and central nervous system. CB2 receptors (CB2R) are primarily found in cells associated with the tissue found in the immune system but are also found in the brain and have been shown to bind with non-psychoactive phytocannabinoids. Studies suggest that the CB2R plays a part in the immune system's regulation of inflammation.

Cannabinoid receptors are G-protein-coupled receptors, which allow them to directly influence the incoming signals. This functions as an “override” signal, which differs from most other cells. As other cells have signal modifiers that can do anything from amplifying to diverging signals, the neuron is “over-riding” those cells. For example, an immune response from the lymphatic system would increase blood flow and the migration of white blood cells to an affected area. The ECS can recognize excess lymphatic signals and, after deciding that there is no longer a need for an increase in inflammation, the cannabinoid receptors in the surrounding immune cells and tissues will begin to bind with cannabinoids to start to slowly reduce the inflammatory response. Cannabinoids permit communication and coordination between different types of cells and are rapidly synthesized and degraded, which therefore suggests that a cannabinoid therapy would be a safer alternative to opiods or benzodiazepines. Cannabinoids, such as THC, can also bind to CB1Rs and CB2Rs to help restore homeostasis. CBD also affects homeostasis but is not believed to bind to receptors the way THC does. It is believed that CBD may inhibit enzymatic deconstruction of endocannabinoids. It is also possible that CBD binds to a receptor that has yet to be discovered.

Over the past decade, primary interest has focused on CB1Rs for their purported role across a range of physiological functions, including directing the psychoactive effect of Δ-9-tetrahydrocannabinol (THC), a phytocannabinoid present in cannabis. CB1Rs are one of the most common G-protein-coupled receptors in the central nervous system, preferentially residing on presynaptic neurons across diverse regions including the neocortex, striatum, and hippocampus. Their widespread distribution allows them to guide a host of functions ranging from cognition, memory, mood, appetite, and sensory responses. Endocannabinoids themselves function as neuromodulators that are released by post-synaptic neurons and bind to the presynaptic CB1Rs to moderate the release of neurotransmitters, such as gamma-aminobutyric-acid (GABA), glutamate, and dopamine. While the specific CB1R function depends on the cell population and region in which they reside, their role in retrograde signaling permits them to regulate signaling activity across cognitive, emotive, and sensory functions, lending therapeutic capacity.

Of the functions that the ECS is involved in, of critical interest, is its influence on the brain reward circuitry, particularly in response to substances of abuse. The rewarding effect of substances of abuse is thought to be primarily mediated by the mesolimbic dopamine pathway, originating from dopaminergic cell bodies in ventral midbrain [ventral tegmental area (VTA)], carrying reward-related information to the ventral striatum [nucleus accumbens (NAc). The acute reinforcing effect of addictive substances is thought to be due to their direct or indirect activation of dopamine neurons along this pathway. The VTA-NAc pathway as such plays a key function in reward assessment, anticipation, and valuation, making it a critical component underlying substance use and addiction.

Dopamine activity is intrinsically tied to cannabinoid activity. CB1Rs are particularly densely located across the striatal regions that mediate reward function (i.e., NAc and VTA), and their regulatory role on the VTA-NAc pathway may be crucial in modulating overall reward tone. Rodent studies have demonstrated that THC increases neuronal firing rates in the VTA, likely through local disinhibition of dopaminergic neurons, by binding to CB1Rs present on glutamatergic and/or GABAergic neurons (although it is prudent to note that THC's capacity to potentiate dopaminergic release differs between rodents and humans). Similarly, other substances of abuse (e.g., opioids, cocaine) have also been demonstrated to potentiate dopaminergic activity via the ECS. For example, alcohol is found to have a downstream potentiation effect on the ECS in rats, such as an increase in endogenous cannabinoid (anandamide and 2-AG) levels and downregulation of CB1R expression. Alcohol-induced dopaminergic release is furthermore dependent on the presence of CB1Rs. Nicotine activates dopamine neurons in the VTA either directly through stimulation of nicotinic cholinergic receptors or indirectly through glutaminergic nerve terminals that are modulated by the ECS. Meanwhile opioid receptors are often co-located with CB1Rs in the striatum and may be modulated by and interact with CB1R activity reciprocally. Only psychostimulants are suggested to act directly on Dopaminergic axon terminals in the NAc, potentially avoiding upstream endocannabinoid involvement in the VTA.

CB1R's role in the motivational and reinforcing effects of rewards has been demonstrated in animal models with CB1R agonists. For example, acute exposure to CB1R agonists (e.g., THC; CP 55,940; WIN 55,212-2; HU 210) augments NAc dopamine transmission, lowers the brain-reward threshold, induces conditioned place preference (CPP), and establishes persistent self-administration of substances of abuse, including cannabis and alcohol. Meanwhile, CB1R antagonists (e.g., rimonabant) have been shown to attenuate reinforcing effects of these substances, blocking the increase of dopamine release in the NAc. While substances of abuse, such as alcohol, stimulants, nicotine and opioids have differing upstream mechanisms of action, the evidence suggest the downstream involvement of the ECS in their reward mechanism.

The ECS, by direct CB1R activity, modulates and is modulated by mesolimbic dopamine activity. While the action of individual substances may differ, they share a common effect of precipitating Dopaminergic activity from the VTA neurons, with this dopaminergic activity mediated by the ECS. It is thus thought that the disruption of endocannabinoid signaling may prove effective in treating SUDs. Nevertheless, it is necessary to note that this is a simplistic understanding, given the potential involvement of non-dopaminergic neurons in the VTA, and additional neuronal circuits including those involving glutamatergic and opioids, that are yet to be fully elucidated.

Unlike many other diseases, pharmacotherapy or behavioral/cognitive therapy alone is unlikely to be sufficient to either restore the damaged system(s) or to prevent relapse and sustain recovery from addiction. Pharmacotherapy alone may only help to reduce the severity of the disorder(s). Current evidence indicates that, to achieve effective treatments and long-term recovery from SUDs, a combination of therapeutic intervention strategies is likely required that include pharmacological treatments and evidence-based behavioral/cognitive therapies (newer therapies using brain stimulation and other nontraditional approaches are also in development).

Despite our extensive understanding of the effects of addiction on behavior and the underlying neurobiology, knowledge remains limited on how the affected biological systems interact with external environmental factors and across the molecular, cellular, and system levels during the development of and recovery from SUDs. The challenge in identifying successful long-term treatments for SUDs is complex because of various factors. Individual differences in responding to treatments are among the known factors common to all SUDs. These differences are reflected in various ways, including genetic determinants (e.g., sex and other forms of genetic heterogeneity), differences in metabolic responses to medications, comorbidity with SUDs (e.g., addicted to alcohol and nicotine or cocaine and other drugs) and with other illness(s) (e.g., depression, HIV infection, and trauma), and the severity and behavioral manifestations of SUDs. Other issues are the motivation and degree of commitment to treatment(s), social environment and support, and the availability and/or ability to afford the cost of treatments. Withdrawal symptoms can be severe and in many cases are so acute as to create an impediment to addiction recovery.

The first stage of detox, acute withdrawal, is marked by physical withdrawal symptoms that can last from a few days and up to two weeks. Acute withdrawal symptoms are the immediate or initial withdrawal symptoms that occur upon sudden cessation or rapid reduction of the use of addictive substances, including alcohol.

Acute withdrawal can produce more dangerous health consequences—even life-threatening complications—if detox isn't completed in a supervised setting. This is especially true, for example, of individuals who are in the acute withdrawal stage of alcohol, benzodiazepines, and barbiturates, as these substances have increased risk of complications without medical supervision, including seizures or coma. Due to the wide range of acute withdrawal symptoms that may occur, and the various addictive substances that may be used, it is preferably to seek medical assistance to achieve lasting recovery and to avoid relapse.

The second stage of detox, known as post-acute withdrawal syndrome (PAWS) occurs as the brain re-calibrates after active addiction. Unlike acute withdrawal, which is primarily physical withdrawal symptoms, the symptoms of post-acute withdrawal are primarily psychological and emotional symptoms. Depending on the intensity and duration of alcohol or other drug use, post-acute withdrawal is known to last many months. Post-acute withdrawal symptoms typically last between one to two years; however, the severity and frequency of symptoms tend to dissipate as times goes by without the use of addictive substances.

Post-acute withdrawal syndrome can be not only discomforting, but symptoms can appear sporadically, making PAWS a driving factor for many individuals to relapse, despite how committed they are to staying clean and sober. Regardless of the addictive substance(s) used, PAWS are typically the same for most individuals in early recovery from SUDs.

While there are many physical symptoms of withdrawal, it also has an emotional side. These emotional symptoms can accompany withdrawal from any substance and are exacerbated by the acuteness of the physical symptoms.

Fortunately, the physical and emotional symptoms of withdrawal are temporary. Effectively managing the symptoms during withdrawal greatly improves the chances of a successful recovery and reduces the chance of relapse.

Cannabinoids, in particular CBD, CBD's acidic precursor CBDA, and acidic derivative thereof are known to be useful in the treatment of nausea and vomiting, seizures, pain, muscle spasms, inflammation, depression, and cachexia. CBD has also long been known to impart beneficial CNS effects as described in Table 1.

TABLE 1
CNS Effects of CBD

| | |
|---|---|
| Anticonvulsant | ++ |
| Antimetrazol | − |
| Anti-electroshock | ++ |
| Muscle Relaxant | ++ |
| Antinociceptive | + |
| Catalepsy | ++ |
| Psychoactive | − |
| Antipsychotic | ++ |
| Neuroprotective antioxidant activity | ++ |
| Antiemetic Sedation | + |
| Appetitive stimulation | − |
| Appetite suppression | ++ |
| Anxiolytic | ++ |
| Bradycardia | + |
| Tachycardia | − |
| Hypertension | − |
| Hypotension | + |
| Anti-inflammatory | ± |

Cannabinoid refers to every chemical substance, regardless of structure or origin, that joins the cannabinoid receptors of the body and brain and that have similar effects to the terpenophenolic compounds produced by the Cannabis Sativa plant. Cannabis Sativa produces between over 100 cannabinoids and about 400 non-cannabinoid chemicals, including terpenes. There are more than 100 terpene compounds in cannabis. Terpenes can have a synergistic effect with cannabinoid and products can be formulated with specific terpenes to enhance beneficial effects. As with cannabinoids, heat can destroy terpenes and their beneficial properties.

Cannabinoids bind to receptor sites throughout the brain (receptors called CB-1) and body (CB-2). Different cannabinoids have different effects depending on which receptors they bind to. For example, THC binds to receptors in the brain whereas CBN (cannabinol) has a strong affinity for CB-2 receptors located throughout the body. Depending on a product's cannabinoid profile, different types of relief are achievable.

In the biosynthetic pathway of cannabinoids in plant tissues, cannabinoids are biosynthesized in an acidic (carboxylated) form. CBGA is the first cannabinoid product in the cannabis plant. THCAA, CBDA, and CBCA are biosynthesized from CBGA following different pathways, each by a particular synthase. Almost no neutral cannabinoid can be found in significant quantities in fresh plant material. However, the carboxyl group is readily lost under the influence of heat or light, resulting in the corresponding neutral cannabinoids such as cannabigerol, cannabidiol, Δ9-THC, and CBC. Δ9-THC and CBD are two key marker cannabinoids in the cannabis plant. Common useful cannabinoids having therapeutic effects are CBDA, CBGA, CBG, CBD, THC-V, CBN, Δ9-THC, Δ8-THC, CBL, CBC and THCAA.

All of the major cannabinoids present in cannabis and hemp are derived from CBGA. Enzymes convert the CBGA into the three major cannabinoid precursor compounds: THCA, CBCA, and CBDA. The decarboxylation of CBDA yields CBD through the following mechanism.

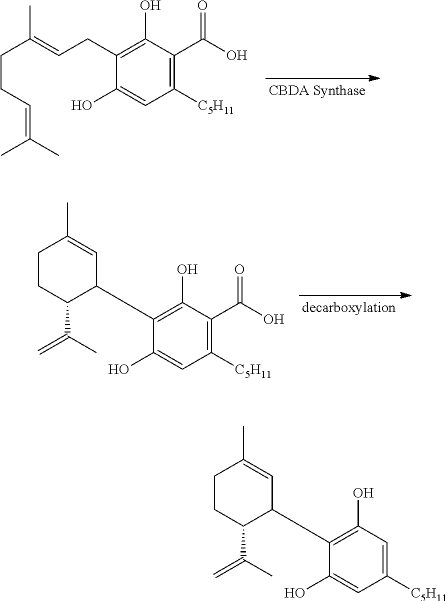

Although not originally considered to be pharmacologically active, research has shown that CBDA closely resembles common non-steroidal anti-inflammatory drugs (NSAIDS) and demonstrated the same COX-2 inhibitor behavior. Further research has shown that CBDA had a far greater affinity to bind to a specific serotonin receptor linked to anti-nausea and anti-anxiety effects. Decarboxylation is induced by heat, therefore cold extraction of CBDA is preferred to improve CBDA yields. The use of cold extraction is also significantly less expensive than the synthesis of CBDA-like compounds and consumes far less energy, making the process friendlier to the environment.

The use of CBDA greatly improves outcomes in addiction recovery treatment by reducing the severity of withdrawal symptoms, thus facilitating a patient's transition through the post-acute withdrawal syndrome stage. A patient's easier transition through the post-acute withdrawal syndrome stage reduces the likelihood of relapse during treatment. The decrease in the rate of relapse has significant health benefits for patients and dramatically reduces the overall cost of treatment frees up much needed manpower and other medical resources. Formulations that include beneficial concentrations of terpenes of interest can further improve outcomes.

Cyclodextrins are cyclic oligosaccharides obtained from starch degradation by cycloglycosyl transferase amylases produced by various bacilli (e.g., Bacillus macerans and B. circulans). Depending on the exact reaction conditions, three main types of cyclodextrins are obtained (α, β, and γ) and each comprises six to eight dextrose units respectively. Cyclodextrins are ring molecules which lack free rotation at the level of bonds between glucopyranose units, they are not cylindrical rather they are toroidal or cone shaped. Cyclodextrins consists of hollow tapered cavity consist of 0.79 nm depth in which the active molecule is incorporated. The primary hydroxyl groups are located on the narrow side whereas the secondary groups are on the wider side. The properties of cyclodextrins can be modified by substituting different functional groups on the cannabidiols rim. Substituting the hydroxyl group of a cyclodextrin by chemical and enzymatic reactions by variety of substituting groups like hydroxypropyl-, methyl-, carboxyalkyl-, thio-, tosyl-, amino-, maltosyl-, glucosyl-, and sulfobutyl ether-groups to β-cyclodextrin can increase the solubility. Solubility of nonpolar solutes occurs due to the nonpolar nature (lipophilic) of the internal cavity of cyclodextrin whereas, the polar nature (hydrophilic) of cyclodextrin's exterior helps in solubilizing the cannabidiol and drug in aqueous solution.

Cyclodextrins are widely soluble in some polar, aprotic solvents, but insoluble in most organic solvents. Although, cyclodextrins exhibit higher solubility in some of the organic solvents than in water, inclusion complexes do not take place in non-aqueous solvents because of the increased affinity of guest molecule for the solvent compared to its affinity for water. Strong acids such as hydrochloric acid and sulfuric acid can hydrolyze cyclodextrins. This hydrolysis rate depends upon temperature and concentration of the acid. Cyclodextrins are stable against bases. The hydrophobic cavity in cannabidiols can partially accommodate low molecular lipophilic drug molecule and polymers. Hydrophilic drug-cyclodextrin complexes are formed by inclusion of lipophilic drug or lipophilic drug molecule in the central cavity. The lipophilic cavity thus protects the lipophilic guest molecule from aqueous environment, while the outer polar surface of the cannabidiol provides the solubilizing effect.

Cyclodextrins offers various advantages in that most are non-toxic and inexpensive. Certain cyclodextrins possess limited application in pharmaceuticals due to low water solubility and safety issues. β-cannabidiol, for example, possesses low solubility and produces hemolytic activity and strong irritancy. However, some β-cyclodextrin derivatives can overcome these shortcomings. Nevertheless, due to its low price, β-cyclodextrin derivatives are widely used in pharmaceutically marketed formulations. The solubility of β-cyclodextrin in water is relatively low whereas its derivative hydroxypropyl-β-cyclodextrin has a significantly higher solubility, Hydroxypropyl-β-cyclodextrin is a widely used derivative of β-cyclodextrin and is used in improving the solubility of hydrophobic drugs with its better aqueous solubility and higher safety. Table 1 represents the natural cyclodextrins and their available derivatives.

TABLE 1
Cyclodextrins and their derivatives

| Cyclodextrins | R | N |
|---|---|---|
| α-Cyclodextrin | H | 4 |
| β-Cyclodextrin | H | 5 |
| γ-Cyclodextrin | H | 6 |
| Carboxymethyl-β-Cyclodextrin | CH2CO2H or H | 5 |
| Carboxymethyl-Ethyl-β-Cyclodextrin | CH2CO2H, CH2CH3 or H | 5 |
| Diethyl-β-Cyclodextrin | CH2CH3 or H | 5 |
| Dimethyl-β-Cyclodextrin | CH3 or H | 5 |
| Glucosyl-β-Cyclodextrin | Glucosyl or H | 5 |
| Hydroxybutenyl-β-Cyclodextrin | CH2CH(CHCH2)OH or H | 5 |
| Hydroxyethyl-β-Cyclodextrin | CH2CH2OH or H | 5 |
| Hydroxypropyl-β-Cyclodextrin | CH2CHOHCH3 or H | 5 |
| Hydroxypropyl-γ-Cyclodextrin | CH2CHOHCH3 or H | 6 |
| Maltosyl-β-Cyclodextrin | Maltosyl or H | 5 |
| Methyl-β-Cyclodextrin | CH3 or H | 5 |
| Random Methyl-β-Cyclodextrin | CH3 or H | 5 |
| Sulfobutylether-β-Cyclodextrin | (CH2)4SO3Na or H | 5 |

Other emulsifiers and water-soluble agents known to those skilled in the art, e.g. lecithin, are also expected to improve bioavailability of cannabinoids as well.

While CBD and CBDA hold therapeutic benefit, a variety of terpenes found in plants have also been used medicinally due their wide array of beneficial properties. Terpenes are the major constituent of the essential oils of plants and are responsible for fragrance, taste, and color. Terpenes can be classified as mono, di, tri, tetra, or sesquiterpene form. A main function of terpenes is to provide protection for the plant from organisms that may feed on it. In humans, terpenes function as anti-malarial agents and have antiviral, anticancer, antidiabetic, and antidepressant benefits

The monoterpenes linalool and beta-pinene have been shown to interact with cannabinoid receptors important in the serotonergic pathway and also in the adrenal glands which play a major part in the management of stress-induced behavior change. In addition to the anti-depressant activity of beta-pinene and linalool, the sesquiterpene beta-caryophyllene has been found to interact with CB2 receptors creating an anti-depressant effect. Terpenes like beta-caryophyllene (BCP), acts as a CB2 receptor agonist making it a novel agent for the prevention and treatment of cancer, diabetes, chronic inflammatory and neurodegenerative diseases, digestive disorders, pain, anxiety and depression.

The combination of cannabinoids and terpenes creates a synergistic entourage effect that modulates the endocannabinoid system and creates a therapeutic overlay of the receptors and enzymes impacting substance use disorder.

The endocannabinoid system has close neurobiological interaction with neurotransmission systems that have important implications for the neural adaptations induced by drug use. CB1 receptors are co-localized with opioid μ opioid receptors in striatal output projection neurons of the nucleus accumbens and dorsal striatum that modulate reward, goal-directed behavior and habit formation relevant to addiction. CB2 receptors have very low expression in the brain generally, but they have been shown to be expressed in dopamine neurons of the midbrain ventral tegmental area and modulate the functional excitability of dopamine neurons central to addiction related behaviors such as drug reinforcement. Stimulation of CB2R in mice models shows an inhibitory influence on cocaine and alcohol self-administration and related conditioned place preference, as well as nicotine place preference behavior.

Furthermore, synergistic analgesia is produced by co-administered cannabinoids/THC and opioids, achieving clinically relevant pain relief at doses that would otherwise be sub-analgesic, thus reducing drug misuse by minimizing dose escalation and the subsequent development of dependence.

The mechanisms of cannabinoid antinociception mimic those of opioid analgesics. Both the CB1R and MOR are G-protein coupled receptors, and agonist-initiated disinhibition of GABA release in the descending pain pathway is an example of overlapping antinociceptive mechanisms between cannabinoids and opioids. The use of the formulation described herein for use in acute, non-severe pain management would allow a substantial reduction in opioid prescription rates, thereby reducing the risks of opioid dose escalation and physical dependence.

### SUMMARY

The formulation described herein is a non-narcotic pharmacologic therapy intended to augment addiction recovery both with and without the use of suboxone or methadone to ease withdrawal symptoms in recovering substance abuse disorder patients. The product may also be formulated with non-psychoactive major and minor cannabinoids, their acidic precursors, and terpenes as adjuncts to orally administered CBDA. Cyclodextrins are used to improve the bioavailability of CBDA and cannabinoids, which have poor solubility in aqueous media. The formulation supports general health and well-being, restores homeostasis, eases the severity of withdrawal symptoms, inhibits opioid misuse (as an analgesic alternative) and decreases the likelihood of relapse.

Also described is a method of improving outcomes in addiction recovery therapies by using the aforementioned formulation to ease withdrawal symptoms upon the removal of suboxone and methadone, and thereby reduce the likelihood of relapse.

### BRIEF DESCRIPTION OF THE DRAWINGS

FIG. 1 illustrates the results expected to be obtained by augmenting the effectiveness of traditional medically supported addiction recovery therapies with an oral preparation of the disclosed formulation.

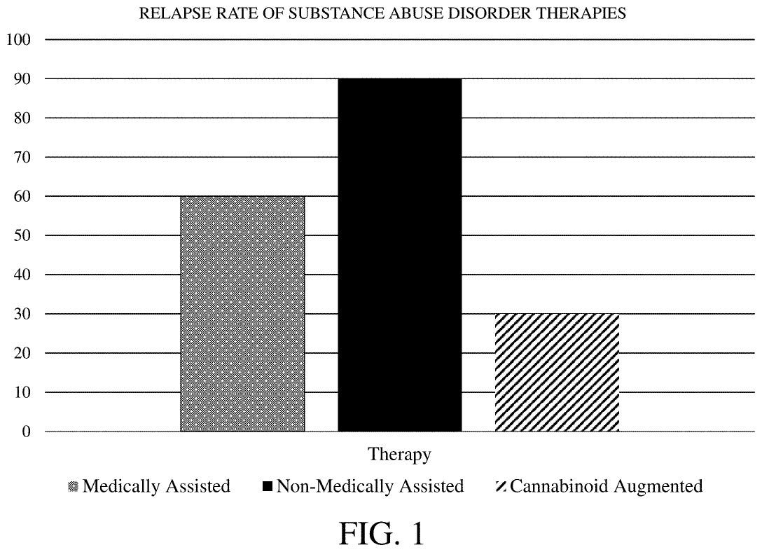

### DETAILED DESCRIPTION

A preparation of efficacious concentrations of at least CBDA, terpenes, and a pharmaceutical adjunct to increase the bioavailability of orally ingested cannabinoids is useful in diminishing the withdrawal symptoms of addictive substances and increasing the effectiveness of substance abuse disorder therapies by reducing the incidence of relapse among patients. An efficacious concentration of CBDA was determined to be at least 50% by mass of the preparation, no more than 49% by mass of decarboxylated cannabinoids (exclusive of Δ-9-THC), and not more than 5% terpenes. It has been found that the decarboxylation of cannabis and its products can increase the yield of neutral cannabinoids. The formulated preparation is formed into a pill using a preferred cyclodextrin as a binder.

In an alternative embodiment, CBN (cannabinol) is utilized to improve efficacy without imparting a psychoactive effect. In a further alternative embodiment, non-psychoactive adjuncts of THC are utilized to improve efficacy. In a still further embodiment, Δ-9-THC (delta-9-tetrahydrocannabinol) is added to the preparation and administered to a patient in a dosage of no more than approximately 0.1 mg of Δ-9-THC per kg of body mass assuming blood has an average density of 0.994 g/ml and the mass of the human body is approximately 10% blood on average (e.g., approximately 5 mg for a 50 kg patient and approximately 10 mg for a 100 kg patient). In formulations in which a psychoactive effect is not contraindicated, such as a step-down intermediary from suboxone, methadone, and naltrexone, dosage of Δ-9-THC can reach up to 1 mg per kg of body mass. Modulation of the psychoactive effect of Δ-9-THC can be achieved by increasing the non-psychoactive cannabinoid loading of the formulation to utilize competitive binding to receptors among cannabinoids to permit an increase in the concentration of Δ-9-THC.

Dosing with preparations containing Δ-9-THC is related to the mass of the patient so as to ensure a maximum blood concentration of Δ-9-THC of less than 1 ng/mL to avoid cognitive impairment and the psychoactive effects of the adjunct. Impairment studies have placed the point of impairment between 1 ng/ml and 5 ng/ml of blood. In a still further embodiment, CBN, THC-v, Δ-8-THC, and Δ-10-THC, and are added as adjuncts to the decarboxylated cannabinoids content of the formulation and may constitute up to 100% of the decarboxylated cannabinoid content. CBN, THC-v and Δ-10-THC are preferred adjuncts because of their lack of a psychoactive effect. THC-v also imparts the added benefit of an appetite suppressant. CBN is a preferred adjunct which avoids the negative connotations of THC and is produced by the degradation of THC. CBN is non-psychoactive and imparts medicinal benefits such as being an antibacterial, a neuroprotectant, an appetite suppressant, an anti-inflammatory, and can help treat glaucoma by reducing intraocular pressure. Depending on a product's formulation and cannabinoid profile, different types of relief are achievable.

In instances where treatment with a psychoactive cannabinoid formulation is preferred, the cannabinoid augmented therapy can consist of a preferred concentration of a psychoactive constituent or a stepwise reduction in concentration of psychoactive constituents during the therapy.

A reduction in relapse among substance abuse disorder patients is expected to be realized by the use of a fully formulated product combined with an agent to improve bioavailability, such as lecithin or cyclodextrins. The product can be formed as a pill using cyclodextrins as a binder. The cyclodextrin quantity utilized is typically 400% of the mass of the active constituents of the product. It is believed that the cyclodextrin acts to nano-encapsulate the product constituents have low water solubility. Orally ingested CBDA has a bioavailability of only approximately 20%. Encapsulating the CBDA in cyclodextrin is expected to increase bioavailability to at least that achieved by intraperitoneal administration, i.e. 80%. Increasing bioavailability through cyclodextrin allows for a more efficient utilization of CBDA than is available without cyclodextrin. This allows the formulator to lower the dose of CBDA and other non-polar constituents and maintain the same effect. The use of emulsifiers and encapsulation increases the bioavailability of the non-polar constituents of the product.

Reduction of the symptoms of withdrawal during the treatment of substance abuse disorder patients is expected to result in a significant improvement in the short-term outcome of both medically assisted therapies and non-medically assisted therapies because a relapse is commonly precipitated by the severity of the withdrawal symptoms. Returning the body to homeostasis by modulating the ECS causes a lessening of the withdrawal symptoms and improves outcomes by reducing the relapse rate from in excess of 90% to less than 50% and potentially less than 30%.

Utilizing the product in long-term therapy of patients with substance abuse disorders also aids in reducing the potential for relapse by maintaining homeostasis by restoring the ECS to its optimal functioning condition which minimizes many triggering events for relapse such as depression, stress, and cravings.

What is claimed is:

1. A method of reducing instances of relapse in substance abuse disorder therapy patients to comprising using an orally administered compounded cannabinoid formulation to alleviate withdrawal symptoms, said cannabinoid formulation containing at least 50% (mass) CBDA and is compounded with an agent to increase bioavailability of non-polar formulation constituents.

2. The method of claim 1, wherein said substance abuse therapy at least one of a medically assisted treatment and a non-medically assisted treatment, wherein said medically assisted treatment further comprises the use of an intermediary drug to facilitate a step down from opiates.

3. The method of claim 2, wherein said intermediary drugs are selected from the group consisting of suboxone, methadone, and naltrexone.

4. The method of claim 1, wherein said cannabinoid formulation further comprises less than 50% (mass) non-CBDA decarboxylated cannabinoids.

5. The method of claim 4, wherein said decarboxylated cannabinoids are selected from the group consisting of CBGA, CBG, CBD, THCV, CBN, Δ9-THC, Δ8-THC, CBL, CBC and THCAA.

6. The method of claim 5, wherein said cannabinoid formulation further comprises less than 5% (mass) terpenophenolic compounds.

7. The method of claim 1, wherein said agent to increase bioavailability is selected from the group consisting of lecithin and cyclodextrins.

8. The method of claim 7, wherein said agent to increase bioavailability is compounded with said cannabinoid formulation at a mass ratio of at least 3:1.

9. The method of claim 4, wherein said compounded cannabinoid formulation is administered with a non-psychoactive dose of Δ9-THC of less than 10 mg per 50 kg body mass to yield a blood concentration of Δ9-THC of less than 1 ng/ml.

10. The method of claim 4, wherein said compounded cannabinoid formulation is administered in a psychoactive dose of Δ9-THC from between 10 mg to 50 mg per 50 kg of body mass.

11. The method of claim 10, wherein said compounded cannabinoid formulation is administered in decreasing concentrations of Δ9-THC.

12. The method of claim 10, wherein said drugs utilized in said medically assisted treatment are selected from the group consisting of suboxone, methadone, and naltrexone.

13. A method of restoring and maintaining homeostasis via the endocannabinoid system comprising using an orally administered compounded cannabinoid formulation, said cannabinoid formulation containing at least 50% (mass) CBDA and is compounded with an agent to increase bioavailability of non-polar formulation constituents.
